(19) United States
(12) Patent Application Publication
(10) Pub. No.: US 2008/0015449 A1
(43) Pub. Date: Jan. 17, 2008

(54) Fiberoptic laser Doppler probe

(76) Inventor: Kaylie David, Franklin, TN (US)

Correspondence Address: MILES & STOCKBRIDGE PC, 1751 PINNACLE DRIVE, SUITE 500, MCLEAN, VA, 22102-3833, US

(21) Appl. No.: 11/730,498

(22) Filed: Apr. 2, 2007

Related U.S. Application Data
(63) Continuation of application No. 60/787,499, filed on Mar. 31, 2006.

(51) Int. Cl. A61B 5/0265; A61B 18/22

(52) U.S. Cl. 600/479; 606/16

(57) ABSTRACT

A laser Doppler flowmetry probe and method for monitoring blood flow in a locus of a tissue medium proximately located at the distal end, the probe having a proximal and distal end and comprising: an optical conductor, having one end located at the distal end, an opposite end located at the proximal end and having a central axis, that guides radiation to said distal end and guides radiation scattered from the tissue medium to the proximal end for providing an indicative of the blood flow in the locus of the tissue medium; and wherein the distal end of the optical conductor is angled with respect to the central axis of the optical conductor so as to provide maximal contact with the tissue.

### FIELD OF THE INVENTION

The present invention relates to a method and apparatus for recording microvascular blood flow in tissue during surgery, giving a measure of real-time tissue perfusion.

### BACKGROUND OF THE INVENTION

The invention is based on the use of laser-Doppler technique (LDF) for measuring the superficial circulation of blood in tissue. This technique is described, for instance, in U.S. Pat. Nos. 3,511,227, 4,109,647, SE 419 678 and the articles “In Vivo Evaluation of Microcirculation by Coherent Light Scattering”, Stern, N. D., Nature, Vol. 254, pp. 56-58, 1975; “A New Instrument for Continuous Measurement of Tissue Blood Flow by Light Beating Spectroscopy”, Nilsson, G. E., Tenland, T. and Oberg, P. A., IEE trans., BME-27, pp. 12-19, 1980, and “Evaluation of a Laser Doppler Flow Meter for Measurement of Tissue Blood Flow”, Nilsson, G. E., Tenland, T. and Oberg, P. A., IEE trans., BME-27, pp. 597-604, 1980.

In principle, this technique involves directing a laser beam onto a part of the tissue and receiving, with the pid of an appropriate photodetector, part of the light scattered and reflected back by that part of the tissue that is irradiated by the laser beam. As a result of the Doppler effect, the frequency of the reflected and scattered light is broadened and the frequency spectrum of the light will thus be broader than the frequency spectrum of the original laser beam, this broadening of the light frequency being due to the influence of movement of blood cells in the superficial part of the irradiated tissue. The extent to which the frequency is broadened and the intensity of light within different parts of this broader frequency spectrum constitute a measurement of the superficial blood circulation in the irradiated part of the examined tissue and can be determined or evaluated by appropriate processing of the photodetector output signal.

The ability to measure real-time tissue perfusion during surgical operations has many applications. One application for measuring real-time tissue perfusion is in the treatment of vestibular schwannomas. Vestibular schwannomas, also known as acoustic neuromas, are benign tumors that grow from the insulating cells, called Schwann cells, of the vestibular (balance) nerve. The incidence in the general population of new diagnosis of vestibular schwannomas is I in 100,000 people. There are two vestibular nerves, along with the cochlear (hearing) nerve and facial nerve (the nerve that moves the muscles of the face), that travel in the internal auditory canal.

The blood supply to these nerves is the labyrinthine artery, which also is the blood supply to the tumor. Most importantly, this artery is the sole blood supply to the inner ear, and disruption of this artery causes deafness. Dissection of the tumor may cause disruption of this artery causing post-operative facial weakness or deafness in patients who had useful hearing pre-operatively. Unfortunately, promontory recordings of hearing and facial function are not possible during surgery. Therefore, there is a need for an intraoperative tool that will provide an indirect measure of hearing and facial function. Measuring the integrity of the microvascular circulation in the facial nerve and cochlea during tumor dissection permits a surgeon to implement dissection maneuvers when blood flow impairment is identified before permanent damage is done. This can improve or save hearing and prevent facial function impairment. Accordingly, proper measurement of microvascular circulation requires a viable measuring method and tool.

Malignant tumors frequently require surgical resection. These surgeries can leave large soft-tissue defects that can seriously impair function and it is imperative to reconstruct these cancer defects. Tissues can be harvested from other parts of the body with the artery and vein that supply it with blood to be used in the reconstruction. The artery and vein are then reattached to arteries and veins near the defect, thereby allowing the harvested tissue or “flap” to maintain blood supply. This procedure is called a “free flap” reconstruction. Once the flap is sewn in to fill the defect, it is imperative to monitor the flap to make sure that the artery or vein has not clotted and obstructing inflow or outflow of blood. If that happens, the flap will not survive and the patient will need to undergo an additional reconstructive surgery. The microcirculation of the flap is monitored using crude methods such as observing the skin color to see if it looks healthy and “pink”. It is also pricked with a small needle to see if it bleeds well. Theses are non-quantifiable methods. A tool that could give objective data concerning the microcirculation would allow for more accurate analysis of the health of the flap and allow for earlier interventions if blood flow is compromised.

### SUMMARY OF THE INVENTION

In accordance with various embodiments of the present invention, a method and apparatus for measuring microvascular circulation are provided. In accordance with a particular embodiment of the present invention, laser Doppler flowmetry (LDF) is used.

Generally, in the practice of the invention, a beam of laser light, carried by a fiber-optic probe, is widely scattered and partly absorbed by the tissue being studied. When light hits moving blood cells, the light undergoes a change in wavelength, which may be referred to as a Doppler shift, while light hitting static objects is unchanged. The magnitude and frequency distribution of these changes in wavelength are directly related to the number and velocity of blood cells but unrelated to their direction of movement. The information is picked up by a returning fiber, converted into an electronic signal and analyzed.

According to the present invention, the laser Doppler technique generally measures blood flow in the very small blood vessels of the microvasculature, such as the low-speed flows associated with nutritional blood flow in capillaries close to the skin surface and the flow in the underlying arterioles and venules involved in regulation of skin temperature. Generally, the tissue thickness sampled is approximately 1 mm, the capillary diameter is approximately 10 microns and the velocity spectrum measurement approximately 0.01 to 10 mm/s. In general, single point measurements give a high temporal resolution (40 Hz data rates are typical) enabling rapid blood flow changes to be recorded, and laser Doppler techniques can provide spatial information and have the ability to average blood flow measurements over large areas. Fiber-optic systems can measure at tissue sites not easily accessible to a laser beam. For example measurements in the brain tissue, mouth, gut, colon, muscle and bone.

According to particular embodiments of the invention, a specially designed Laser Doppler Fiber-optic (LDF) probe and a method of contacting the probe with a tissue site are provided, in order to record microvascular blood flow in tissues during surgery and give a measure of real-time tissue perfusion. The system and method of the present invention are particularly adapted to measure microvascular flow in the facial nerve and cochlea during surgery for removal of, e.g., vestibular schwannoma tumors. The system and method of the invention also enable measurement of microvascular flow in free flap tissues during reconstruction surgeries for cancer defects. In general, the invention, functions as a neurologic or cancer reconstruction surgical tool and offers a dynamic and non-invasive device and method for measurement of microvascular flow.

Experimentation with standard LDF probes for measuring perfusion rates have proved impractical for application to the facial nerve since all standard LDF probes are flat end probes that are not suitable for placement during surgery. The flat end was not capable of making adequate tissue contact and thus unable to record microvascular blood flow in tissues.

In accordance with an embodiment of the present invention, a uniquely designed probe is provided which allows the use of standard LDF measurement systems. The unique probe of the invention enables the utilization of the angle at which the facial nerve enters the boney canal under the anatomic region called the transverse crest to access the necessary tissue. The crux of the invention resides in a LDF probe, the tissue-contacting tip of which is angled. The incorporation of the correct angle in the probe tip enables the probe to be securely fixed to the nerve surface under study. The angled probe can also be placed on the cochlea during middle fossa approaches for acoustic neuroma tumor removal. The angle is necessary to allow adequate probe contact with the convex cochlea surface. The modified probe may be held in place with a standard instrument holder. This allows accurate readings of perfusion rates.

Generally, it is helpful to use a drop of saline solution on the end of the modified probe before securing it to the surface of the nerve. This helps to complete the sealing of the angled fiber end and the surface of the nerve tissue, given that there may not be an exact fit. For example, the saline may serve to seal any air gaps and prevent any light distortion.

Stretching or compression of the nerve or artery during surgery can affect blood flow. Knowing the effect on perfusion will allow a surgeon to take preemptive measures. In addition, knowing the microcirculation status of a free flap after it has been inset into the defect will allow for an accurate measurement of flap viability.

In accordance with other embodiments of the present invention, a modified probe may have varying angles depending on the artery or specific feature being analyzed. The modified probe may also be adapted to fold into transferred tissue to take direct arterial readings to ensure the implanted tissue is properly perfused.

Another embodiment of the present invention includes a novel method and device that provides several advantages over existing laser Doppler flowmetry (LDF) methods and devices. For example, these advantages include:
(A) Stable placement of the probe during surgery;
(B) Real-time measurements of cochlear blood flow, which correlates with hearing;
(C) Real-time measurements of free-flap blood flow, which correlates with flap viability and survival;
(D) Malleable probe for placement over irregular surfaces; and
(E) Small diameter for measurement of narrow caliber nerves.

In general, this technology is extremely useful to neurologists for measuring cochlear microvascular blood flow during the middle fossa hearing preservation procedure related to the internal auditory canal. It is also useful for measuring microvascular blood flow in the facial nerve during the trans labyrinthine approach to the internal auditory canal. Likewise, similar uses in other surgical procedures where measuring microvascular blood flow is useful are also contemplated. One such surgical procedure is free-flap reconstruction of surgical defects.

Generally, in the case of acoustic neuroma removal, it is not known whether there is any loss of facial muscle control until the patient is examined post-operatively. In the case of free tissue transfer, the effectiveness of the surgery is determined by rudimentary tests such as pinpricks to induce bleeding when the patient is examined postoperatively. In acoustic neuroma surgeries in which hearing preservation is attempted, the current intra-operative hearing measurements have a lag time of 2-3 minutes. It is therefore not possible using that measurement tool to know if you are impairing hearing in real-time. In the case of free tissue transfer, the effectiveness of the surgery is determined by rudimentary tests such as pinpricks to induce bleeding when the patient is examined postoperatively. In contrast, embodiments of the present invention provide a unique methodology for instant readings, during a surgical procedure, that alert a surgeon to any difficulty and allows him the opportunity to take corrective measures should the flow rate become interrupted.

Embodiments of the present invention also have potential non-neurological applications, including, but not limited to, for example:

(A) Oncological Surgery—Cancers of the head and neck often require the extirpation of large amounts of tissue, resulting in major functional deficits. Free tissue transfer surgery takes tissues from other parts of the body with the artery and veins that service that tissue and transfer them into the defect. The artery and veins are anastamosed to their counterparts in the vicinity of the defect. It is imperative during the anastomosis and tissue-insetting phase of the surgery to have adequate perfusion of the transferred tissue. Unfortunately, this is currently assessed by crude means like pricking the tissue with a needle to see if it bleeds, or applying pressure and looking for capillary refill in the tissue. These are unquantifiable techniques and fraught with inter-observer error. In accordance with embodiments of the present invention, a fiber-optic laser Doppler (FLD) probe can be placed on the tissue to obtain a quantifiable measurement of microvascular perfusion during surgery;

(B) Neurosurgical Aneurysm Surgery—The LDF probe may be placed on intracranial aneurysms to assess blood flow in collateral vessels prior to clipping of the aneurysm. In general, this will increase intra-operative safety and allow for a quantifiable assessment of patency of feeding vessels; and

(C) Vascular Surgery—The LDF probe may be used to assess tissue perfusion after bypass surgery in the limbs. It may give a quantifiable value of perfusion of tissue distal to the graft, which is an indirect measure of graft patency.

### BRIEF DESCRIPTION OF THE DRAWINGS

FIG. 1 shows an LDF probe within its metal sheath on a facial nerve, in accordance with an embodiment of the present invention.

FIG. 2 shows an LDF probe within its metal sheath on a facial nerve, in accordance with another embodiment of the present invention.

FIG. 3 shows an LDF probe within its metal sheath on the cochlea, in accordance with an embodiment of the present invention.

FIG. 4 is an elevational view of a probe according to the invention.

FIGS. 5 and 6 are graphical depictions of results of tests employing the probe of the invention.

### DETAILED DESCRIPTION OF THE INVENTION In accordance with an embodiment of the present invention, a

Laser Doppler Fiber-optic (LDF) probe is provided to measure microvascular blood flow in tissue during surgery to obtain a measure of real-time tissue perfusion.

As tissue structures may comprise small, non-planar surfaces (for example, the facial nerve is approximately 1 mm in diameter), in general, a very small, flexible probe is needed to get a good reading from the tissue. In accordance with an embodiment of the present invention the probe includes a specially designed LDF probe that has a fiber-optic cable with an angled, polished tip. This generally allows for maximal contact with the tissue surface during surgery. The fiber-optic cable may be placed through a malleable tube that may be held in place using a standard surgical retractor holder.

Unlike standard LDF probes, an embodiment of the present invention calls for the fiberoptic cable to have an angled tip and for placing the cable through a suction tube. The metal tube allows for the fiber-optic cable to be bent into a variety of favorable shapes so it can be placed in full contact with the tissue site.

In the present embodiment of the invention, the fiber-optic cable generally has a diameter of from about 0.4 mm to about 1 mm. The length is approximately 1.5 meters. It will be understood by those skilled in the art that the ultimate parameters of the probe of the invention will vary, depending upon the application contemplated. The tip is machined to provide even seating on the desired structure. The distal 1-3 mm of the probe is bent 30°, 45° or 60° depending on the configuration of the tissue. However, other angles below, between and above these angles are also possible. The tip is polished to further define an angle. The proximal end of the fiber optic may be inserted into a “master probe” supplied by an outside vendor, or have connectors at the end of the probe that directly interface with the laser Doppler machine without the need of the interposed “master probe”. The fiber optic can be sterilized.

In accordance with another embodiment of the present invention, the outer catheter may include, but is not limited to, a metal sheath with a caliber of 20 French (Fr) to provide rigid support for the fiber-optic cable, but be malleable enough to allow precise placement of the probe on the desired tissue. The outer catheter may be approximately 3 cm, more or less, in length. The LDF probe may be inserted through the outer catheter and secured to it with sterile adhesive tape, and the outer catheter may be sterilized. The outer catheter may also be permanently affected to the fiber optic during the manufacturing process.

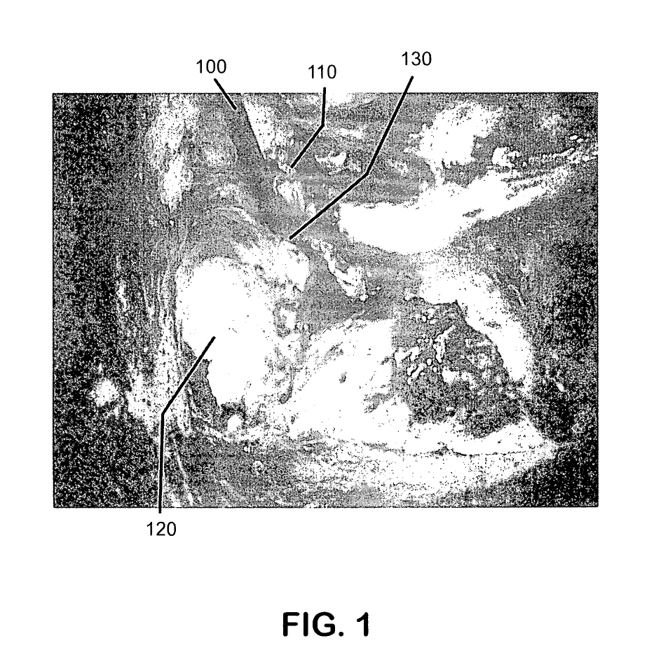

FIG. 1 shows an LDF probe 100 within its metal sheath on a facial nerve 110, in accordance with an embodiment of the present invention. A tumor 120 is shown adjacent to facial nerve 110 and a distal end of LDF probe 100. In accordance with an embodiment of the present invention, LDF probe 100 may be specifically designed to have a 0.4 mm diameter and a 30° taper 130 to accommodate the channel location needed for accurate measurement of, for example, a 1.0 mm diameter nerve at a 30° incline. In this embodiment, the LDF probe may be fitted directly to the nerve surface, allowing a surgeon to accurately monitor blood flow in real time. Knowing the integrity of the microvascular circulation in the tissue during surgery is useful in allowing the surgeon to alter his dissection maneuvers before any permanent damage is done.

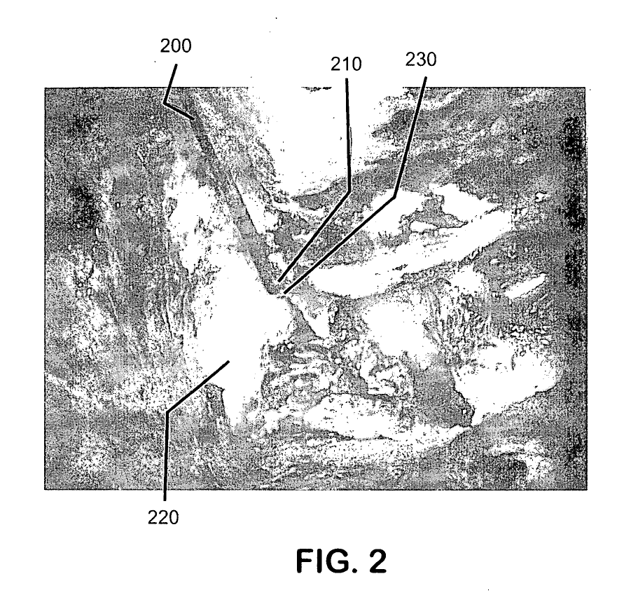

FIG. 2 shows an LDF probe 200 within its metal sheath on a facial nerve 210, in accordance with another embodiment of the present invention. A tumor 220 is shown adjacent to facial nerve 210 and a distal end of LDF probe 200. In accordance with an embodiment of the present invention, LDF probe 200 may be specifically designed to have a 0.4 mm diameter and a 30° taper 230 to accommodate the channel location needed for accurate measurement of, for example, a 1.0 mm diameter nerve at a 30° incline. In this embodiment, the LDF probe is fitted directly to the nerve surface, allowing a surgeon to accurately monitor blood flow in real time. Knowing the integrity of the microvascular circulation in the tissue during surgery is useful in allowing the surgeon to alter his dissection maneuvers before any permanent damage is done.

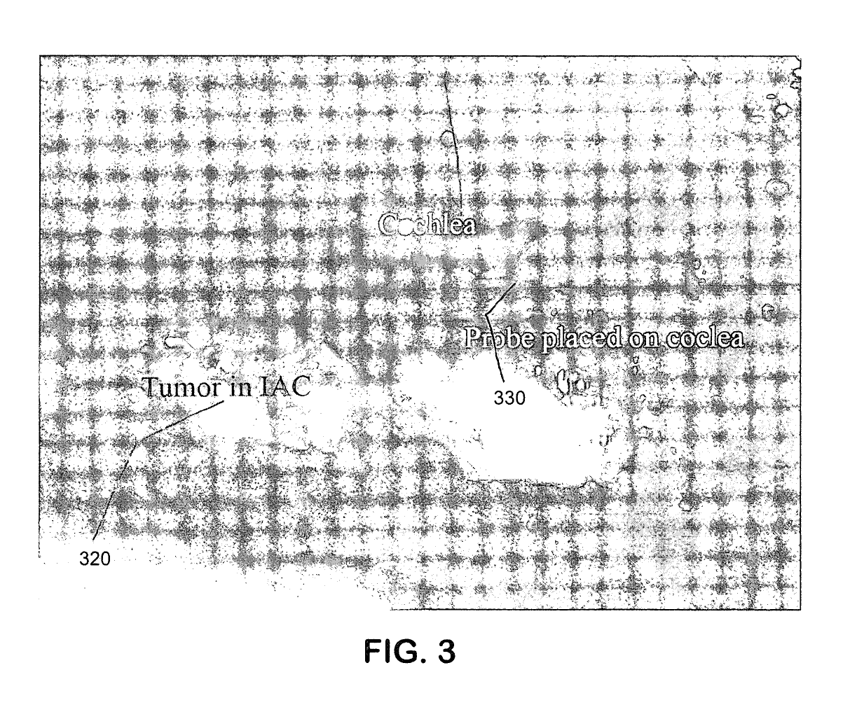

FIG. 3 shows an LDF probe 300 within its metal sheath on cochlea 310, during a middle fossa surgery, in accordance with another embodiment of the present invention. A tumor 320 is shown adjacent to facial nerve 310 and a distal end of LDF probe 300.

In accordance with an embodiment of the present invention, LDF probe 300 may be specifically designed to have a desired diameter and angled tip 330 to accommodate the channel location needed for accurate measurement of, for example, a 1.0 mm diameter nerve at a 30° incline.

It will be understood by those skilled in the art that any standard LDF instrumentation may be employed in the practice of the invention. In the examples a Periflux System 5000 (Perimed)—780 nm semiconductor laser—output less than 1 mW was utilized: perfusion units at 0.1 second intervals—continuous until tumor removed—cauterization of major vessels flagged on output—output divided into areas by cauterization events.

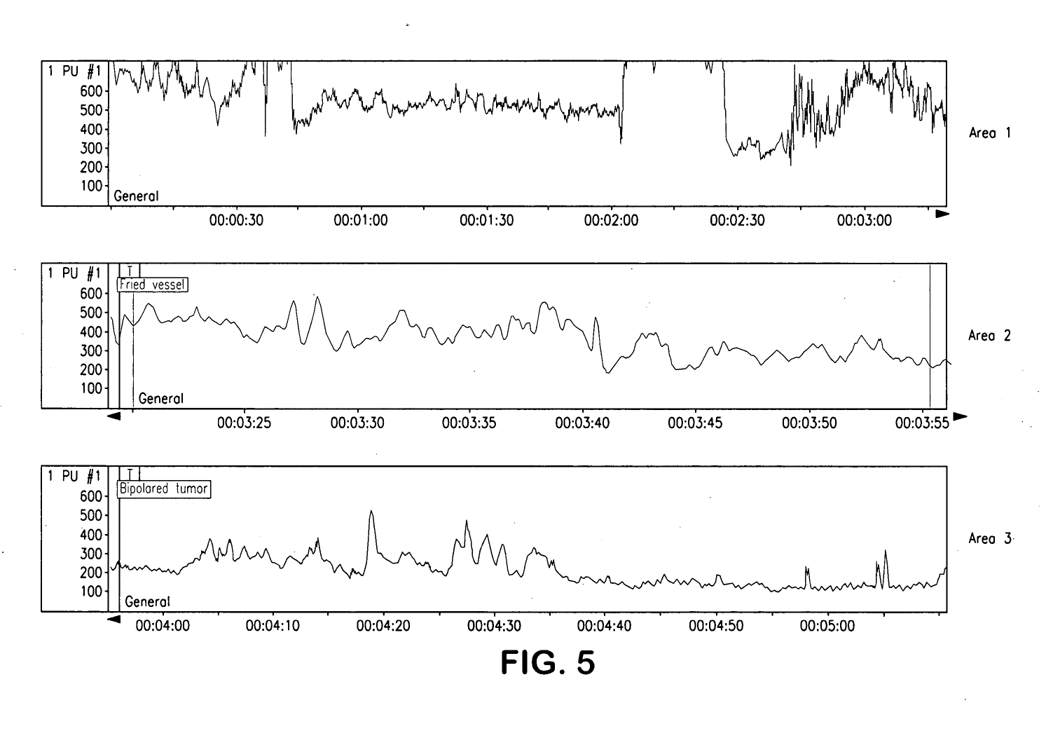

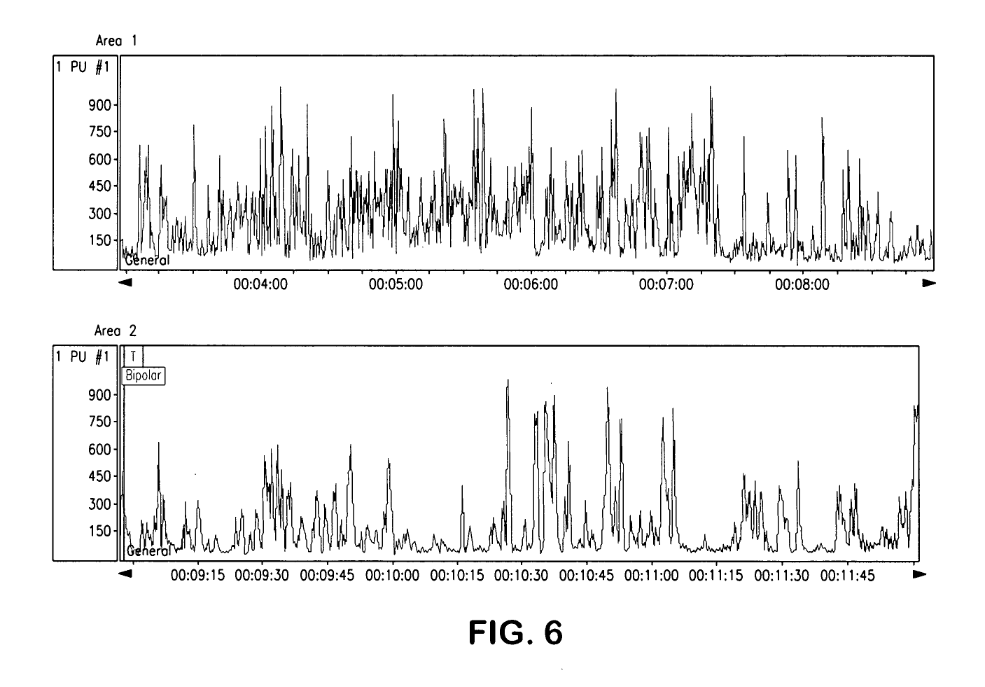

Two patients were included in the study; ages 52 and 48, with tumor sizes 0.9 and 2.1 cm:
Patient 1—One major vessel cauterized—Slope of area 1=−0.25—Slope of area 2=0.1; Percent change from area 1 to 2=−28.3% Significant difference between two areas. See FIG. 6.
Patient 2—Two major vessels cauterized—Slope of area 1=−0.05—Slope of area 2=−6.4—Slope of area 3=−2.5. Percent change from area 1 to 2=−37% Percent change from area 2 to 3=−44% Percent change from area 1 to 3=−64.7% Significant difference between three areas. See FIG. 5.

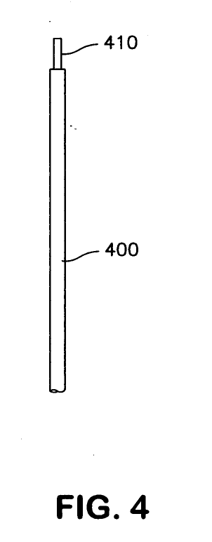

FIG. 4 is an elevational view of the probe of the invention wherein LEF probe 400 within its metal sheath is shown. A distal end of the LDF probe 400 specifically designed to have a desired diameter and angled tip 410.

In accordance with one or more other embodiments of the present and LDF probe may include an angle that depends on the artery or specific feature being analyzed. In accordance with another embodiment of the present invention, the LDF probe may be modified to accommodate another and potentially larger application, for example that of measuring microvascular flow in real time during and after free tissue transfer surgery. Generally, the device may be adapted to fold into the transferred tissue to take direct arterial readings to ensure that the implanted tissue is properly perfused.

In accordance with another embodiment of the present invention, a method of measuring microvascular blood flow in tissue during surgery may include, but is not limited to, placing a drop of saline on the tip of the fiber portion of the probe, in order to avoid reflections; placing the probe on the tissue site prior to performing surgery; and recording perfusion units at 0.1 second intervals until completion of the surgical procedure. During measurement, the probe may be held in place with a standard instrument holder.

In accordance with another embodiment of the present invention, and LDF probe may be used for relative measurements. In accordance with another embodiment of the present invention, if the LDF probe can be placed at one location, to continually monitor a given site for the duration of the critical period, the changes noted may be directly related to absolute volume flow changes in the sampled tissue, when proper monitoring technique is maintained.

1. A laser Doppler flowmetry probe, having a proximal and distal end, for monitoring blood flow in a locus of a tissue medium proximately located at the distal end, said probe comprising:
an optical conductor, having one end located at said distal end, an opposite end located at said proximal end and having a central axis, that guides radiation to said distal end and guides radiation scattered from said tissue medium to said proximal end for providing an indicative of the blood flow in the locus of the tissue medium; and
wherein said distal end of said optical conductor is angled with respect to said central axis of said optical conductor so as to provide maximal contact with said tissue.

2. The probe of claim 1 wherein said tissue is nerve tissue.

3. The probe of claim 2 wherein said nerve tissue is the cochlear or facial nerve.

4. The probe of claim 1 wherein said distal end is angled between about 30° and about 60°.

5. The probe of claim 1 wherein said distal end is angled about 30°.

6. The probe of claim 1, wherein said optical conductor is housed in a sheath that will accommodate said angled distal end.

7. The probe of claim 1, wherein said sheath comprises an elongated housing member having proximal and distal ends and at least one elongated passage with proximal and distal openings, said optical conductor extending within said elongated passage.

8. The probe of claim 7 wherein said optical conductor extends through said proximal opening to provide a proximal section of said optical conductor outside of said elongated housing member.

9. The probe of claim 8, said proximal end of said optical conductor being configured for coupling to an optical radiation source.

10. The probe of claim 7 wherein said optical conductor extends through said distal opening to provide a distal section of said optical conductor outside of said elongated housing member.

11. A method monitoring blood flow by laser Doppler flowmetry in a locus of a tissue medium, comprising the steps of:
inserting the probe of claim 1 into a subject such that maximum contact is made by said angled distal end thereof with said tissue;
illuminating said tissue by generating an optical signal which is guided by said laser Doppler flowmetry probe to be incident on said tissue;
receiving a backscattered optical signal from said tissue and guided by said laser Doppler flowmetry probe; and
processing said backscattered optical signal to provide a signal indicative of blood flow within said tissue.

12. A system for assessing patient status, comprising:
monitoring means for receiving said signal indicative of blood flow produced by the method of claim 10 and processing means adapted to acquire from said monitoring means, and for processing, said signal to assess patient status.

13. The system for assessing patient status of claim 11, further comprising:
a laser Doppler flowmetry probe, having a proximal and distal end, for monitoring blood flow in a locus of a tissue medium proximately located at the distal end, said probe comprising:
an optical conductor, having one end located at said distal end and an opposite end located at said proximal end, that guides radiation to said distal end and guides radiation scattered from said tissue medium to said proximal end for providing an indicative of the blood flow in the locus of the tissue medium; and
wherein said distal end of said probe is angled with respect to the axis of said probe so as to provide maximal contact with said tissue.

14. In a system for monitoring blood flow in a locus of a tissue medium to determine blood perfusion in blood vessels of a body organ, said system comprising:
a laser light source (1) generating a laser beam (2), means for directing said laser beam (2) onto said tissue (3), the improvement wherein said means for directing said laser beam comprises the laser Doppler flowmetry probe of claim 1.
